(19) United States
(12) Patent Application Publication
(10) Pub. No.: US 2023/0065462 A1
(43) Pub. Date: Mar. 2, 2023

(54) Pharmaceutical composition and use thereof

(71) Applicant: HENAN TAIFENG BIOLOGICAL TECHNOLOGY CO., LTD., Kaifeng City, Henan Province (CN)

(72) Inventors: Wei SHA, Kaifeng City, Henan Province (CN); Yanjun ZHANG, Kaifeng City, Henan Province (CN); Jia SONG, Kaifeng City, Henan Province (CN); Ting LI, Kaifeng City, Henan Province (CN)

(21) Appl. No.: 17/793,918

(22) PCT Filed: Jan. 19, 2021
(86) PCT No.: PCT/CN2021/072630

§ 371 Date: Jul. 19, 2022

(30) Foreign Application Priority Data
Jan. 19, 2020 (CN) 202010058725.1

(51) Int. Cl. A61K 9/00; A61P 7/04; A61P 1/00; A61K 31/194; A61K 31/7048; A61K 31/192

(52) U.S. Cl. CPC A61K 9/0019; A61P 7/04; A61P 1/00; A61K 31/194; A61K 31/7048; A61K 31/192

(57) ABSTRACT

A pharmaceutical composition. The pharmaceutical composition comprises citric acid, paeoniflorin and gallic acid, wherein the weight ratio of citric acid, paeoniflorin and gallic acid is about 100:(1˜8):(0.1˜5). Also provided is use of the pharmaceutical composition in the treatment of rectal hemangioma and in the treatment of hemorrhoids in patients with leukemia and hemophilic.

### TECHNICAL FIELD

The disclosure belongs to the field of medical technology. Specifically, the disclosure relates to a pharmaceutical composition and the use of the pharmaceutical composition in the treatment of rectal hemangioma and hemorrhoids, especially in patients not suitable for surgical treatment, such as the treatment of hemorrhoids in patients with leukemia and hemophilia.

### BACKGROUND ART

Rectal hemangioma is a non-single-cell benign tumor, of which the main cell components include endothelial cells, fibroblasts, adipocytes, etc. The etiology of rectal hemangioma is still unclear, and it is generally considered to be a hamartoma originating from the remnants of the mesoderm embryo, and is a very rare benign vascular lesion. The clinical manifestations of rectal hemangioma lack specificity, of which the main clinical manifestations are that patients suffer from recurrent painless hematochezia, and some patients have discomforts such as abdominal pain, perianal swelling. Rectal hemangioma can recurrently attack, has a long course of disease and progressive aggravation, and often has a clinical manifestation of chronic anemia. Since some of the clinical manifestations are similar to hemorrhoids, rectal hemangioma is easy to be misdiagnosed as hemorrhoids or rectal inflammation.

Now, the main treatment of rectal hemangioma is injection sclerotherapy. The principle of action of sclerosing agent is that: after acting on local tissues, sclerosing agent first destroys endothelial cells, causing tissues to undergo swelling, degeneration, necrosis, and inflammatory cell infiltration to different degrees, and then fibrosis, fibroblast proliferation, causing blood vessels to undergo compression, occlusion, or embolization, and then causing tissues to shrink and fall off, thereby gradually regressing the hemangioma. Although the injection sclerotherapy has a significant therapeutic effect, simple operation and no complicated equipment, there is a lot of bleeding during and after the operation, and rectal hemangioma is easy to relapse. In addition, the injection sclerotherapy can also cause local phlebitis, inflammatory reactions, and even serious adverse reactions such as infection and necrosis.

A hemorrhoid is a common disease in the anal area, can attack at any age, but with age, the incidence gradually increases. There are two main theories about the etiology of hemorrhoids: one is the theory of varicose veins, in which hemorrhoids are venous clusters formed by congestion, expansion and flexion of the venous plexus under the mucosa of the lower part of rectum and under the skin of the anal canal; the other is Thomson's theory of anal pads shifting down which is currently widely accepted, in which hemorrhoids are originally the normal anatomical structure of the anal canal, that is, the vascular pads, and are a dentate line and a circular spongy tissue band 1.5 cm above the dentate line.

The treatment of hemorrhoids includes surgical treatment and non-surgical treatment, of which surgical treatment is the main one, including thrombotic external hemorrhoid dissection, traditional hemorrhoidectomy, circumcision of hemorrhoid and PPH surgery; and non-surgical treatment includes topical medication, oral drug therapy, physical therapy, rubber band ligation, and injection therapy. However, the conventional injection therapy is to inject sclerosing agent around the submucosal venous plexus to cause inflammation and fibrosis, thereby compressing the varicose veins, but the injection of sclerosing agent into the mucosal layer will cause necrosis.

Hemophilia is a group of bleeding disorders with inherited coagulation dysfunction, of which common features are that the generation of active thromboplastin has some problems, the blood coagulation time is prolonged, the tendency to bleeding after minor trauma is lifelong, and severe patients can also have “spontaneous” bleeding without obvious trauma. The severely ill patients will bleed as long as the skin is slightly wounded. In addition to the general symptoms of hemorrhoids in patients with hemophilia and hemorrhoids, the risk of bleeding is particularly serious. Patients with hemophilia and hemorrhoids are prone to repeated heavy bleeding after hemorrhoid surgery, which makes the patients very scared, causing severe psychological and physical injuries to the patients;

and repeated operations are required, which also brings additional financial burdens. In addition, the colostomy also brings inconvenience to patients' life and care. Therefore, the risk of surgical treatment for such patients is relatively high, and more serious patients are prone to death, and they need to be injected with expensive clotting factors.

Leukemia is a malignant clonal disease of hematopoietic stem cells. Because of mechanisms such as uncontrolled proliferation, impaired differentiation, and blocked apoptosis, lots of leukemia cells accumulate in bone marrow and other hematopoietic tissues, and infiltrate other non-hematopoietic tissues and organs, while inhibiting normal hematopoietic function. Leukemia and hemophilia are similar blood diseases, so the treatment principle is to eliminate the leukemia cell population and control the proliferation of leukemia cells. Therefore, like the case of hemophilia with hemorrhoids, the case of leukemia with hemorrhoids also poses a bleeding risk. When surgical treatment is performed on patients with leukemia and hemorrhoids, on one hand, due to the problem of blood clotting function, it will cause continuous bleeding; on the other hand, due to the decline of immune function, it may cause perianal infection, thereby spreading to systemic infection, or it may induce sepsis to threaten the life.

Surgical treatment often causes great damage to the patient, resulting in a decrease in resistance, causing undesirable consequences such as complications. In addition to the above-mentioned patients with coagulation disorders such as hemophilia and leukemia who are not suitable for surgical treatment, medical practice has proved that the surgery are also not suitable to patients with a bleeding tendency (patients with coagulation disorders (such as aplastic anemia, leukemia or hemophilia), patients receiving antithrombotic treatment (such as patients receiving cardiac stent surgery or cardiac pacemaker surgery, or patients with myocardial infarction or cerebral infarction), patients with hypertension, diabetes, portal hypertension, renal dysfunction, myasthenia gravis, and immune deficiency (such as acquired Immune Deficiency Syndrome (AIDS), systemic lupus erythematosus), and patients undergoing chemoradiotherapy. For these patients, if they have rectal hemangioma or hemorrhoids at the same time, they should not undergo surgery.

Now, there is no reliable treatment method for rectal hemangioma and hemorrhoids in patients who are not suitable for surgical treatment. Therefore, there is an urgent need to find a therapy without surgery.

### DISCLOSURE

The inventors conducted research on the basis of the prior art and founded that a three-component composition (especially injection) of citric acid, gallic acid and paeoniflorin can treat the above-mentioned diseases instead of surgical treatment.

The composition of citric acid, gallic acid and paeoniflorin is different from the necrosis or hardening mechanism of sclerosing agent, which “shrinks” the entire rectal hemangioma or hemorrhoid through a series of changes of tissue protein coagulation, lysis, absorption, and capillary angiogenesis, won't leave induration and other sequelae after treatment, and meanwhile, also greatly reduces the risk of bleeding. In the composition, the organic acids of citric acid and gallic acid make blood vessels constrict, decrease blood flow and blood vessel permeability, shorten blood clotting time, and promote platelet aggregation at the damaged blood vessel, coagulate and degenerate of large blood vessel walls and interstitial tissue, and disintegrate tissue; and paeoniflorin promotes blood circulation to remove blood stasis, inhibits acute inflammation, promotes the proliferation of interstitial fibrocytes, induces macrophages to clear the disintegrated tissues, promotes capillaries proliferation, and inhibits the fibrosis of hypertrophic scars.

In addition, the inventors also founded that a specific ratio of citric acid, gallic acid and paeoniflorin, as well as a specific ratio of gallic acid and paeoniflorin can produce a synergistic effect and achieve a better therapeutic effect.

Therefore, in one aspect, the disclosure provides a pharmaceutical composition containing citric acid, paeoniflorin and gallic acid in a weight ratio of about 100:(1˜8):(0.1˜5).

In another aspect, the disclosure provides a preparation comprising the pharmaceutical composition of the disclosure and a pharmaceutically acceptable excipient.

In another aspect, the disclosure provides a use of the pharmaceutical composition of the disclosure in a preparation of a medicament for a treatment of rectal hemangioma or hemorrhoids.

In another aspect, the disclosure provides the pharmaceutical composition of the disclosure, which is used to treat rectal hemangioma or hemorrhoids.

In another aspect, the disclosure provides a method for treating rectal hemangioma or hemorrhoids, which comprises administering the pharmaceutical composition of the disclosure or the preparation of the disclosure.

### BEST MODE

### Definition

Herein, all technical and scientific terms have the same meanings as commonly understood by those skilled in the art to which the disclosure belongs, unless otherwise specified, but in case of conflict, the definitions in this specification shall prevail.

As used in the description and claims, the singular forms “a”, “an” and “the (said)” include plural forms, unless the context clearly dictates otherwise.

The percentages (%) in this description are weight percentages (wt %), unless otherwise specified.

All numerical values or expressions referring to component amounts used in the description and claims should be understood to be modified by “about” in all cases. When referring to a quantity or a numerical range, the term “about” means that the quantity or numerical range referred to is an approximation within the error of statistical experiments. Therefore, the quantity or numerical range may be within, for example, ±5% of the stated quantity or numerical range.

All ranges referring to the same components include endpoints, which can be independently combined. Since these ranges are continuous, they include every value between the minimum and maximum values. It should also be understood that any numerical range cited in this application is intended to include all sub-ranges within that range.

The abbreviations used in the disclosure have the usual meanings in the fields of medicine, chemistry, and biology.

### Active Ingredient

### Citric Acid

Citric acid is also called ciTCMLIBic acid, and its chemical name is 2-hydroxypropane-1,2,3-tricarboxylic acid. Citric acid is widely distributed in nature, and natural citric acid exists in fruits such as lemons, citrus, pineapples and bones, muscles and blood of animals. Citric acid is an edible acid, mainly used in the food industry, such as sour agents, solubilizers, buffers, antioxidants, deodorants, flavor enhancers, gelling agents, and toners. Some foods have good tastes after adding citric acid, which can promote appetite. Thus, citric acid is often used in jams, beverages, cans and candies.

### Paeoniflorin

Paeoniflorin, derived from Paeonia lactiflora root, peony root, purple peony root, has a following chemical structure:

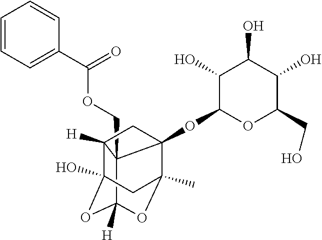 [SMILES: C[C@@]12C[C@@]3(O)O[C@@H](O1)[C@]1(COC(=O)c4ccccc4)[C@H]3C[C@@]12O[C@@H]1O[C@H](CO)[C@@H](O)[C@H](O)[C@H]1O]

The paeoniflorin has a very low toxicity, and there is no obvious untoward effect under normal circumstances. The paeoniflorin can resist the oxidative stress damage of tissue cells, inhibit the activation of astrocytes, and enhance the protection of nerves. Since the paeoniflorin has obvious antagonistic effects on the damage of striatum and substantia dopaminergic nerve cells, and can improve bradykinesia, it can be used to treat brain diseases such as Alzheimer's disease, Parkinson's disease and epilepsy. In addition, the paeoniflorin can also resist autoimmune diseases such as tumors, rheumatoid arthritis and ankylosing spondylitis. Animal experiments also proved that the paeoniflorin can significantly reduce blood sugar levels and has an important protective effect on heart and lung cells.

### Gallic Acid

Gallic acid is also called trihydroxybenzoic acid. Its chemical name is 3,4,5˜trihydroxybenzoic acid. It is widely present in plants such as Rheum palmatum, Eucalyptus robusta, Cornus officinalis, is a polyphenolic compound that exists in nature, and has a wide range of applications in food, biology, medicine, chemical and other fields.

Gallic acid has various biological activities such as anti-inflammatory, anti-mutation, anti-oxidation and anti-free radicals; and meanwhile, it has anti-tumor effects, which can inhibit the metastasis of mast cell tumors, thereby prolonging the survival period; it is also a candidate drug which is relatively suitable for killing trypanosome; it has a protective effect on the liver, which can resist carbon tetrachloride-induced physiological and biochemical changes in the liver; and it can induce endothelial-dependent contraction and endothelial-dependent relaxation by inhibiting the production of endothelial NO.

The mechanism of action of the three components is as follows: the organic acids of citric acid and gallic acid make blood vessels constrict, blood flow decrease, blood vessel permeability decrease, blood clotting time shorten, promoting platelet aggregation at damaged blood vessels, coagulation and degeneration of large blood vessel walls, coagulation and degeneration of interstitial tissues, and disintegration of tissues; while paeoniflorin promotes blood circulation to remove blood stasis, inhibits acute inflammation, promotes the proliferation of interstitial fibroblasts, induces macrophages to clear the disintegrated tissues, promotes capillary proliferation, and inhibits hypertrophic scar fibrosis.

### Pharmaceutical Composition and Formulation

In the composition of the disclosure, the three components are mixed in a specific ratio to produce biological effects such as reducing the number of capillaries, proliferating collagen fibers, and killing vascular endothelial cells. When the weight ratio of citric acid, gallic acid and paeoniflorin is in a specific range, the above biological effects are more obvious.

In a specific embodiment of the disclosure, the weight ratio of citric acid, gallic acid and paeoniflorin is about 100:(1˜8):(0.1˜5); in another specific embodiment, the weight ratio of citric acid, gallic acid and paeoniflorin is about 100:(1.5˜7.5):(0.2˜4); in yet another specific embodiment, the weight ratio of citric acid, gallic acid and paeoniflorin is about 100:(2˜7):(0.5˜3.5); in yet another embodiment, the weight ratio of citric acid, gallic acid and paeoniflorin is about 100:(2˜6.5):(0.5˜3); and in yet another specific embodiment, the weight ratio of citric acid, gallic acid and paeoniflorin is about 100:(2˜6):(1˜2).

Among the above specific ratios, the specific ratio of gallic acid and paeoniflorin is also critical. Therefore, in a specific embodiment of the disclosure, the weight ratio of paeoniflorin to gallic acid is about (1˜6):1; in another specific embodiment, the weight ratio of paeoniflorin to gallic acid is about (1˜5):1; in yet another specific embodiment, the weight ratio of paeoniflorin to gallic acid is about (1˜4):1; in yet another specific embodiment, the weight ratio of paeoniflorin to gallic acid is about (1.5˜3.5):1; and in yet another specific embodiment, the weight ratio of paeoniflorin to gallic acid is about (2˜3):1.

In a more specific embodiment, the weight ratio of citric acid, gallic acid and paeoniflorin is about 100:4:1.5, 100:5.6:2, 100:2.2:1, 100:3.6:1.8, 100:3.6:1.3, 100:2:1.2, 100:6.2:2, 100:2.5:0.8, and 100:5.3:2.6, preferably about 100:4:1.5, 100:5.6:2 and 100:2.2:1, and particularly preferably about 100:4:1.5.

### Injection

The pharmaceutical composition of the disclosure is preferably used in the form of injection.

Injections refer to sterile solutions (including emulsions and suspensions) made of medicines for injection into the body, and sterile powders or concentrated solutions that are prepared into solutions or suspensions immediately before use, and mainly include liquid injection, powder for injection, tablet for injection, etc.

A liquid injection is also called a parenteral solution, commonly known as a “water injection”, which is a preparation made by formulating a drug into a solution (aqueous or non-aqueous), suspension or emulsion and filling the resultant into an ampoule or multi-dose container. Generally, a water-soluble drug is required to achieve quick-acting effects after injection, so it is often formulated into an aqueous solution or a water composite solution (for example, additionally adding ethanol, propylene glycol, glycerin, etc. into the aqueous solution). Some drugs are not suitable to be made into aqueous solutions, such as the drugs being insoluble in water or the medicines prolonging their efficacy after injection, etc., which can be made into oil solutions, water or oil suspensions, or emulsions. But these injections are generally only for intramuscular injection.

A powder for injection, commonly known as a “powder injection”. Some drugs have poor stability and are easy to decompose and deteriorate after being made into solutions. Such drugs generally can be used by filing sterile powdered drugs for injection into ampoules or other suitable containers via aseptic manipulation and dissolving or suspending the resultant in a suitable solvent before use.

A tablet for injection refers to a molded tablet or machine-pressed tablet made of drugs by aseptic manipulation, which are dissolved in water for injection before use and are used for subcutaneous or intramuscular injection.

### Preparation Method of Injection

a. Weighing 3.75 g of gallic acid, 10 g of paeoniflorin, and 250 g of citric acid in a clean room with a temperature of 20˜25° C. and a relative humidity of 45%˜65%;

b. Adding the above three raw materials to the dosing device;

c. Adjusting the temperature in the dosing device to 40˜50° C., and adding water for injection into the dosing device until the total weight of the liquid medicine being 100 kg with a stable air pressure and a stable temperature in the dosing device;

d. Stirring the liquid medicine for 3˜10 minutes, adjusting the temperature in the dosing device to 15˜25° C., and filtering the liquid medicine;

e. Aseptically encapsulating the product obtained in d into 10 mL/bottle injection, obtaining the injection after being checked for leaks at 100° C., sterilized with 60 Kpa under negative pressure for 20 minutes, inspected, and packaged.

### Treatment Method and Use

The “method” or “use” of the disclosure refers to administering the pharmaceutical composition of the disclosure to a subject who has a corresponding disease or a tendency of the disease, with the purpose of imparting a therapeutic effect, such as curing, alleviating, changing, affecting, improving or preventing the symptoms or tendencies of the aforementioned diseases. Those skilled in the art can easily determine the specific effective dose according to the type of disease to be treated, the route of administration, and the use of excipients, since the dose may vary due to the simultaneous use of other drugs.

### Disease

### Rectal Hemangioma

Rectal hemangioma is a congenital non-hereditary disease, which is rare in clinical practice. Because of non-progressive growth characteristics, rectal hemangioma is generally considered to be a hamartomas rather than a tumor neoplasm. The disease usually occurs in the rectum. It more commonly occurs in adolescents aged 10 to 20, with more men than women. According to the morphology of blood vessels in hemangioma, it can be divided into three categories: capillary hemangioma, cavernous hemangioma, and mixed hemangioma.

The cause of rectal hemangioma is still unclear. It is generally believed that the rectal hemangioma is originated from an embryonic vascular hamartoma bud, and the embryonic vascular hamartoma bud can be led to tumor-like proliferation under the action of certain factors. Capillary hemangioma causes slow bleeding, and cavernous hemangioma causes massive bleeding, which will become gradually severe and frequent. Polyp-type colonic hemangioma can cause intestinal obstruction due to intussusception, and volvulus occurs in some cases. Extensive diffuse rectal hemangioma manifests as systemic coagulation mechanism disorder, aggravating intestinal bleeding, accompanied by thrombocytopenia, hypofibrinogenemia, decreased blood coagulation factors V and VIII levels, etc. After the rectal hemangioma is removed, it can be restored to normal.

The clinical manifestations of rectal hemangioma vary with the type, size and location of the hemangioma. For those with a history of hemangioma, mucosal or skin of similar lesions, and signs of hematochezia, anemia, and intestinal obstruction, especially children or young persons, the possibility of this disease should be considered. Fiber endoscopy, lower gastrointestinal angiography, and mesenteric angiography, etc., can be used as a basis for lesion location and diagnosis. This disease should be distinguished from internal and external hemorrhoids, colorectal inflammation, ulcers, rectal villous adenoma, rectal polyps, and anorectal prolapse.

Rectal hemangioma requires active treatment. Among untreated patients, 40% of patients die from bleeding caused by hemangioma. According to the patient's general condition, the size and location of the hemangioma, non-surgical treatment or surgical treatment can be used.

1. Non-Surgical Treatment

Non-surgical treatment includes sclerosing agent injection, freezing, diathermy or electrocautery, electrocoagulation and other therapies to make the tumor fibrosis, thereby reducing the tumor and stopping bleeding. It is suitable for a patient with low-position hemangioma, a patient with poor general condition, and a patient with rectal hemangioma who cannot tolerate surgical resection. The hemostatic effect is better in the short term, but the long-term effect and radical cure are poor, and the recurrence rate is high.

2. Surgical Treatment

The best way to treat rectal hemangioma is surgical removal. Partial intestinal resection is feasible for colonic hemangioma; small rectal hemangiomas can be resected under local submucosal, preserving the muscle layer and serous membrane; upper rectal lesions undergo anterior rectal resection via abdomen; and for diffuse or multiple types of middle and lower rectal lesions, the best surgical method is combined rectal-abdominal-perineum resection, but since most patients are young, it is difficult to accept a permanent abdominal colostomy, which causes postoperative sexual dysfunction, so it should be avoided.

### Hemorrhoid

A haemorrhoid (commonly known as a hemorrhoid) is a common disease located in the anal area, can attack at any age, but with age, the incidence gradually increases.

There are two main theories about the etiology of hemorrhoids. The first is the theory of varicose veins, which believes that hemorrhoids are venous clusters formed by congestion, expansion and flexion of the venous plexus under the mucosa of the lower part of rectum and under the skin of the anal canal. However, the currently widely accepted theory is Thomson's theory of anal pad shifting down, which believes that hemorrhoids are originally the normal anatomical structure of the anal canal, that is, the vascular pad, and are a dentate line and a circular spongy tissue band 1.5 cm above the dentate line. Only when the anal pad tissue is abnormal and combined with symptoms can it be called hemorrhoids and need treatment. The purpose of treatment is to relieve symptoms, not to eliminate hemorrhoids.

Asymptomatic hemorrhoids do not need treatment; symptomatic hemorrhoids do not need radical treatment; and non-surgical treatment is the main treatment.

1. Non-Surgical Treatment

(1) Local medication treatment: it has been widely used, and the medications include suppositories, ointments and lotions, most of which contain Chinese herbal ingredients.

(2) Oral drug treatment: it generally uses drugs for the treatment of varicose veins.

(3) Injection therapy: sclerosing agent is injected around the submucosal venous plexus to cause inflammation and fibrosis, thereby compressing the varicose veins; but injection of sclerosing agent into the mucosal layer will cause necrosis.

(4) Physical therapy: laser therapy, cryotherapy, direct current therapy and copper ion electrochemical therapy, microwave thermal coagulation therapy, infrared coagulation therapy are less used.

(5) Rubber band ligation: the root of the hemorrhoids is ligated to block the blood supply to make the hemorrhoids fall off and necrotic; it is more suitable for huge internal hemorrhoids and fibrotic internal hemorrhoids.

2. Surgical Treatment

(1) Dissection of thrombotic external hemorrhoid: it is suitable for patients whose pain does not relieve or whose mass does not shrink after conservative treatment of thrombotic external hemorrhoids.

(2) Traditional hemorrhoidectomy: it can be referred to as external stripping and internal ligation.

(3) Hemorrhoid circumcision (Whitehead operation): it is the classic operation in textbooks which can easily lead to anal stenosis, which is rarely used in clinical practice.

(4) PPH operation: it can be referred to as procedure for prolapsing hemorrhoids, created by the Italian doctor Longo, and was promoted in 1998. It is mainly suitable for prolapsed type III-IV mixed hemorrhoids, circular hemorrhoids, and partially bleeding serious II internal hemorrhoids. The mechanism of PPH in the treatment of prolapsed hemorrhoids: 2 to 3 cm of lower rectal mucosa and submucosal tissue are circularly excised of to restore the normal anatomical structure, that is, the anal pad is returned; the resection of the submucosal tissue blocks the blood flow from the supra hemorrhoidal artery to the hemorrhoid area, so that postoperative hemorrhoid atrophies. As compared with traditional hemorrhoidectomy, PPH operation has shorter operation time, less postoperative pain, faster recovery, and fewer complications, but the equipment is more expensive.

### Patient

### Patient who is not Suitable for Surgery

Due to the bleeding tendency, slow wound healing, secondary infection, poor physical condition, etc., some patients are not suitable for surgery. These patients mainly include: patients with bleeding tendency (patients with coagulation dysfunction (such as patients with aplastic anemia, leukemia or hemophilia), patients receiving antithrombotic therapy (such as patients receiving cardiac stent surgery or cardiac pacemaker surgery, or patients with myocardial infarction or cerebral infarction)), patients with hypertension, diabetes, portal hypertension, renal dysfunction, myasthenia gravis or immunodeficiency (such as AIDS, systemic lupus erythematosus), or patients receiving chemoradiotherapy.

Patient with Bleeding Tendency

A bleeding tendency refers to a clinical manifestation in which a skin or mucosa is spontaneously bleeding, or the bleeding is not easy to stop spontaneously when the tiny blood vessels suffer minor trauma.

The causes of bleeding tendency are generally divided into three categories: abnormal blood coagulation function, platelet abnormalities, and abnormal blood vessel wall function. Coagulation dysfunction refers to a bleeding disorder caused by a deficiency or abnormal function of coagulation factors, which mainly includes hemophilia, vitamin K deficiency, and coagulation abnormalities caused by severe liver disease. Platelet abnormalities mainly include thrombocytopenia, platelet dysfunction, etc. Abnormal blood vessel wall function mainly includes Henoch-Schonlein purpura.

In addition, some other diseases and their treatment process can also cause bleeding tendency. For example, one of the common symptoms of leukemia patients is thrombocytopenia, followed by a tendency to blood. Another example is that after surgeries of various cardiovascular and cerebrovascular diseases such as coronary heart disease, after heart bypass surgery, after coronary stent surgery, after heart valve replacement surgery, after large vessel disease surgery, etc., long-term use of antithrombotic drugs such as aspirin and warfarin is required for treatment, but the use of antithrombotic drugs has led to bleeding tendency.

Hemophilia with Hemorrhoid

Hemophilia is a group of bleeding disorders with inherited coagulation dysfunction, of which the common features are the disorder of active thromboplastin production, a prolonged coagulation time, a lifelong tendency to bleeding after minor trauma, and severe patients can also experience “spontaneous” bleeding without obvious trauma. Hemophilia is a group of sex-linked recessive bleeding disorders, and is clinically divided into two types, i.e., hemophilia A and hemophilia B, which are caused by gene mutations in coagulation factor VIII and coagulation factor IX, respectively.

After a patient with hemophilia suffers from hemorrhoid, its treatment is more difficult than ordinary people. The patient with hemophilia has a higher risk of surgical treatment, which can easily lead to bleeding, and the patient with more serious conditions is likely to die. When treating hemorrhoid, the patient with hemophilia needs to inject expensive coagulation factors; and when undergoing surgical treatment, he/she also needs to prepare fresh plasma in addition to coagulation factors so as to prevent postoperative bleeding.

Leukemia with Hemorrhoid

Leukemia is a malignant clonal disease of hematopoietic stem cells. Due to mechanisms such as uncontrolled proliferation, differentiation disorder, and obstructed apoptosis, clonal leukemia cells accumulate in bone marrow and other hematopoietic tissues, and infiltrate other non-hematopoietic tissues and organs, while inhibiting normal hematopoietic function. Clinically, various degrees of anemia, hemorrhage, infection and fever, enlarged liver, spleen and lymph nodes, and bone pain can be seen.

Leukemia and hemophilia are similar to blood diseases. One common feature is that a leukemia patient has thrombocytopenia, blood vessel damage, blood clotting dysfunction, etc., which leads to the risk of bleeding. Nearly 40% of leukemias have bleeding as the early manifestation. Bleeding can occur in various parts of the body, common with unexplained skin bruises, bleeding oral cavity, bleeding nasal cavity, bleeding gums, menorrhagia, etc. Blurred retina often indicates that the patient has fundus hemorrhage; and severe headache with nausea and vomiting often indicates that the patient has intracranial hemorrhage.

If patients with leukemia undergo surgical treatment of hemorrhoids, on the one hand, due to their coagulation function, the surgical treatment will cause continuous bleeding; on the other hand, the surgical treatment may cause perianal infections, spreading to the whole body, or inducing sepsis and be life-threatening.

### Method of Administration

The specific method of administration is as follows:

(1) Placing a patient at a side lying position, a folding knife position and a bladder lithotomy position and other treatment positions, performing routine disinfection, and then anal local anesthesia or anal canal anesthesia with 0.5-1% of lidocaine;

(2) When using an injection, diluting the product with 0.5-1% of lidocaine at the volume ratio of the drug to lidocaine being 1:(0.8˜1.2);

(3) For stage I and II internal hemorrhoid and varicose mixed hemorrhoid, exposing each hemorrhoid under an anoscope, and inserting a needle into the hemorrhoid obliquely;

(4) Retracting the anoscope to expose the hemorrhoid, in which the injection method for stage III internal hemorrhoid is the same as that for stage I and II internal hemorrhoid;

(5) Administering about 10-20 ml to each patient at a time, with an average of 15 ml and the maximum dosage of no more than 40 ml; and

(6) Generally giving only one injection to each patient.

In one embodiment, the pharmaceutical composition of the disclosure is administered to a patient with rectal hemangioma. In another embodiment, the pharmaceutical composition of the disclosure is administered to a patient with hemorrhoid. In yet another embodiment, the pharmaceutical composition of the disclosure is administered to a patient with rectal hemangioma or hemorrhoid who is not suitable for surgical treatment. In yet another embodiment, the pharmaceutical composition of the disclosure is administered to a patient with rectal hemangioma or hemorrhoid who is prone to bleeding. In yet another embodiment, the pharmaceutical composition of the disclosure is administered to a patient with rectal hemangioma or hemorrhoid who has hemophilia or leukemia. In yet another embodiment, the pharmaceutical composition of the disclosure is administered to a patient with rectal hemangioma or hemorrhoid who is receiving antithrombotic therapy.

In a specific embodiment of the abovementioned embodiment, when administering the pharmaceutical composition of the disclosure, the patient does not stop the original drug treatment, such as antithrombotic drugs. In another specific embodiment, when administering the pharmaceutical composition of the disclosure, the patient does not need to supplement clotting factors or undergo blood transfusion. In yet another specific embodiment, when administering the pharmaceutical composition of the disclosure, the patient's coagulation factor VIII activity is 30% or more, for example, 50% or 80%.

### Specific Embodiment

The disclosure relates to the following technical solutions:

Scheme 1: a pharmaceutical composition containing citric acid, paeoniflorin and gallic acid in a weight ratio of about 100:(1˜8):(0.1˜5), preferably about 100:(1.5˜7.5):(0.2˜4), more preferably about 100:(2˜7):(0.5˜3.5).

Scheme 2: the pharmaceutical composition of scheme 1, wherein the weight ratio of paeoniflorin to gallic acid is about (1˜6):1, preferably about (1˜5):1, more preferably about (1˜4):1.

Scheme 3: the pharmaceutical composition of scheme 1, wherein the weight ratio of citric acid, paeoniflorin and gallic acid is about 100:(2˜6.5):(0.5˜3), and the weight ratio of paeoniflorin to gallic acid is about (1.5˜3.5):1.

Scheme 4: the pharmaceutical composition of scheme 1, wherein the weight ratio of citric acid, paeoniflorin and gallic acid is about 100:(2˜6):(1˜2), and the weight ratio of paeoniflorin to gallic acid is about (2˜3):1.

Scheme 5: the pharmaceutical composition of scheme 1, wherein the weight ratio of citric acid, paeoniflorin, and gallic acid is about 100:4:1.5, 100:5.6:2, 100:2.2:1, 100:3.6:1.8, 100:3.6:1.3, 100:2:1.2, 100:6.2:2, 100:2.5:0.8, 100:5.3:2.6, preferably about 100:4:1.5, 100:5.6:2 or 100:2.2:1, especially preferably about 100:4:1.5.

Scheme 6: a preparation comprising the pharmaceutical composition according to any one of schemes 1-5 and a pharmaceutically acceptable excipient.

Scheme 7: the preparation of scheme 6, which is an injection.

Scheme 8: the preparation of scheme 7, which comprises the pharmaceutical composition and water for injection.

Scheme 9: the injection according to Scheme 7 or 8, which is in a 5 mL or 10 mL dosage format and contains (132±0.5)mg or (264±0.5)mg of the pharmaceutical composition, respectively.

Scheme 10: a use of the pharmaceutical composition of any one of schemes 1-5 in the preparation of a drug for treating rectal hemangioma or hemorrhoid.

Scheme 11: the use of scheme 10, wherein the drug is used for a patient who is not suitable for surgery.

Scheme 12: the use of scheme 11, wherein the patient who is not suitable for surgery includes a patient with bleeding tendency, hypertension, diabetes, portal hypertension, renal dysfunction, myasthenia gravis, or immunodeficiency, or a patient who is receiving chemoradiotherapy.

Scheme 13: the use of scheme 12, wherein the patient with bleeding tendency includes a patient with coagulation dysfunction (such as a patient with aplastic anemia, leukemia or hemophilia), or a patient receiving antithrombotic therapy (such as a patient receiving cardiac stent surgery or cardiac pacemaker surgery, or a patient with myocardial infarction or cerebral infarction).

Scheme 14: the use of scheme 12, wherein the patient with immunodeficiency includes a patient with AIDS or systemic lupus erythematosus.

Scheme 15: the use of any one of schemes 10-14, wherein the drug is used to treat rectal hemangioma.

Scheme 16: the use of any one of schemes 10-14, wherein the drug is used to treat hemorrhoid in a patient with coagulation dysfunction (such as aplastic anemia, leukemia, or hemophilia).

Scheme 17: the pharmaceutical composition of any one of schemes 1-5 or the preparation of any one of schemes 6-9, which is used to treat rectal hemangioma or hemorrhoid.

Scheme 18: a use of the pharmaceutical composition or the preparation of scheme 17, which is used to treat a patient who is not suitable for surgery.

Scheme 19: a use of the pharmaceutical composition or preparation of Scheme 18, wherein the patient who is not suitable for surgery includes a patient with bleeding tendency, hypertension, diabetes, portal hypertension, renal dysfunction, myasthenia gravis, or immunodeficiency, or a patient who is receiving chemoradiotherapy.

Scheme 20: a use of the pharmaceutical composition or preparation of scheme 19, wherein the patient with bleeding tendency includes a patient with coagulation dysfunction (such as aplastic anemia, leukemia or hemophilia), or a patient receiving antithrombotic therapy (such as a patient receiving cardiac stent surgery or cardiac pacemaker surgery, or a patient with myocardial infarction or cerebral infarction).

Scheme 21: a use of the pharmaceutical composition or preparation of Scheme 19, wherein the patient with immunodeficiency includes a patient with AIDS or systemic lupus erythematosus.

Scheme 22: a use of the pharmaceutical composition or preparation of any one of schemes 17-21, which is used to treat rectal hemangioma.

Scheme 23: a use of the pharmaceutical composition or preparation of any one of schemes 17-21, which is used to treat hemorrhoid in a patient with coagulation dysfunction (such as a patient with aplastic anemia, leukemia or hemophilia).

Scheme 24: a method for treating rectal hemangioma or hemorrhoid in a patient, including administering the pharmaceutical composition of any one of schemes 1-5 or the preparation of any one of schemes 6-9.

Scheme 25: the method of scheme 24, wherein the patient is a patient who is not suitable for surgery.

Scheme 26: the method of scheme 25, wherein the patient who is not suitable for surgery includes a patient with bleeding tendency, hypertension, diabetes, portal hypertension, renal dysfunction, myasthenia gravis, or immunodeficiency, or a patient who is receiving chemoradiotherapy.

Scheme 27: the method of Scheme 26, wherein the patient with bleeding tendency includes a patient with coagulation dysfunction (such as a patient with aplastic anemia, leukemia or hemophilia), a patient receiving antithrombotic therapy (such as a patient receiving cardiac stent surgery or cardiac pacemaker surgery, or a patient with myocardial infarction or cerebral infarction).

Scheme 28: the method of scheme 26, wherein the patient with immunodeficiency includes a patient with AIDS and systemic lupus erythematosus.

Scheme 29: a method for treating rectal hemangioma in a patient, including administering the pharmaceutical composition of any one of schemes 1-5 or the preparation of any one of schemes 6-9.

Scheme 30: a method for treating hemorrhoid in a patient with coagulation dysfunction (such as aplastic anemia, leukemia, or hemophilia), including administering the pharmaceutical composition of any one of schemes 1-5 or the preparation of any one of schemes 6-9.

Scheme 31: the method of any one of schemes 24-30, wherein an administered pharmaceutical composition or preparation is an injection in water for injection, and wherein a drug concentration is 22-30 mg/mL, preferably 24-27 mg/mL, and preferably 26.5 mg/mL.

Scheme 32: the method of scheme 31, wherein the injection is used after being diluted with 0.5%-1% of lidocaine at a volume ratio of 1:(0.8˜1.2), preferably at a volume ratio of 1:1.

Scheme 33: the method of scheme 31 or 32, wherein the injection is injected to the patient 1-3 times, and preferably 1 time.

Scheme 34: a method for treating hemorrhoid in a patient with coagulation dysfunction, including administering the pharmaceutical composition of any one of schemes 1-5 or the preparation of any one of schemes 6-9, including the following steps:

(1) Placing the patient at the side lying position, the folding knife position and the bladder lithotomy position and other treatment positions, performing routine disinfection, and then anal local anesthesia or anal canal anesthesia with 0.5-1% of lidocaine;

(2) When using the injection, diluting the product with 0.5-1% of lidocaine at the volume ratio of the drug to lidocaine being 1:(0.8˜1.2);

(3) For stage I and II internal hemorrhoid and varicose mixed hemorrhoid, exposing each hemorrhoid under an anoscope, and inserting a needle into the hemorrhoid obliquely;

(4) Retracting the anoscope to expose the hemorrhoid, in which the injection method for stage III internal hemorrhoid is the same as that for stage I and II internal hemorrhoid;

(5) Administering about 10-20 ml to each patient at a time, with an average of 15 ml and the maximum dosage of no more than 40 ml; and

(6) Generally giving only one injection to each patient.

Scheme 35: the method of scheme 34, wherein the patient is a patient with aplastic anemia, leukemia, or hemophilia.

### EXAMPLE

### Example 1

### Cockscomb Hemangioma Model Experiment

### 1. Materials

Animals: 88 Leghorn chickens of clean grade and raised under uniform feeding conditions in the same batch, male, weighing 1000˜1500 g, 10˜12 weeks old, provided by the Animal Experimental Center of Qinglongshan Animal Breeding Farm in Jiangning, Nanjing. The cockscomb is required to be plump, smooth, ruddy, and no lesion. All animals are reared in the same laboratory according to the requirements of clean grade, and they can eat and drink freely. The experimental process complies with the People's Republic of China “Regulations on the Management of Laboratory Animals” and ethical requirements.

Test drugs: each component of the drug composition is commercially available. Citric acid, paeoniflorin, and gallic acid are mixed at the ratio described in the following table to obtain compositions 1-9 of groups A-I, respectively. Then, each composition was dissolved in water for injection to obtain an injection with a concentration of 13.2 mg/mL. The positive control group used lauromacrogol parenteral solution (Specification: 10 mL: 100 mg, Shaanxi Tianyu Pharmaceutical Co., Ltd.) as a sclerosing agent, and was prepared into a 5 mg/ml injection with water for injection before use.

TABLE 1
The distribution ratio (weight ratio) of each group in the pharmaceutical

| | | Citric | | Gallic | Paeoniflorin/ |
|---|---|---|---|---|---|
| Group | Composition | acid | Paeoniflorin | acid | gallic acid |
| Group | Composition 1 | 100 | 4 | 1.52 | 2.63 |
| A | | | | | |
| Group | Composition 2 | 100 | 5.65 | 1.96 | 2.89 |
| B | | | | | |
| Group | Composition 3 | 100 | 2.22 | 0.96 | 2.31 |
| C | | | | | |
| Group | Composition 4 | 100 | 3.64 | 1.82 | 2 |
| D | | | | | |
| Group | Composition 5 | 100 | 1.56 | 1.27 | 1.23 |
| E | | | | | |
| Group | Composition 6 | 100 | 2.04 | 1.22 | 1.67 |
| F | | | | | |
| Group | Composition 7 | 100 | 6.25 | 1.95 | 3.2 |
| G | | | | | |
| Group | Composition 8 | 100 | 2.52 | 0.8 | 3.15 |
| H | | | | | |
| Group | Composition 9 | 100 | 5.28 | 2.64 | 2 |
| I | | | | | |

### 2. Method

### 2.1 Animal Grouping

The clean grade of male Leghorn chickens were selected and randomly divided into a blank control group, a positive control group and a pharmaceutical composition groups A-I, a total of 11 groups, each with 8 chickens. Four points were selected for the cockscomb of each cock as the drug injection point, and the cockscomb was observed, photographed, and sampled before the experiment and 3 days, 7 days, 14 days, and 28 days after the experiment.

### 2.2 Model Building

The experimental cocks were anesthetized through intramuscular injection of 0.1 mg/kg Sumianxin, fixed, and cleaned, and after being disinfected, each cockscomb was marked with gentian violet to form 4 circles with a diameter of 1 cm in turn on one side of the each cockscomb from front to back, and was injected at the center of each circle: the blank control group was injected with 0.5 mL of solvent (water for injection), the positive control group was injected with 0.5 mL of 5 mg/ml lauromacrogol injection, and the groups A-I were injected with 0.5 mL of the corresponding 13.2 mg/mL injection of compositions 1-9, respectively, and the standard is that the surface tissue of the cockscomb turns white and produces a skin hill. As the thickness of the cockscomb was 8˜10 mm, only one side of the cockscomb is injected subcutaneously, and the non-injected side of the contralateral side was used as the self-control group. After the injection, the cocks were continued to raise under uniform conditions, eaten and drunk freely until the material was obtained.

### 2.3 Indicator Observation

### 2.3.1 General Observation

Before drug injection, the general condition of the cockscomb, including shape, color, thickness, surface condition, etc., were observed and recorded with photos. After the drug injection, the observation was performed every day, and included: the color change of the cockscomb, whether the epidermis has white spots, whether the edema occurs, whether tissue necrosis occurs on the surface, whether scab occurs, whether scar occurs, and whether the scab falls off, and the time distribution of fall off. The observation were recorded by taking photos at four time points, i.e., 3 days, 7 days, 14 days, and 28 days after the drug injection, and meanwhile, the experimental cock was anesthetized and the tissues at the injection site were taken and examined by light microscope.

### 2.3.2 HE Staining

The tissues at the injection site were taken, fixed in 10% neutral formaldehyde fixative for 48 hours, embedded in paraffin, sectioned, stained with conventional HE, and examined by light microscope at 3 days, 7 days, 14 days, and 28 days after drug injection, respectively.

### 2.3.3 Cell Apoptosis Detection

The TUNEL method is used for cell apoptosis detection including cell detection and tissue specimen detection. The principle is that deoxyuridine triphosphate is connected to the 3′-OH end of broken DNA in apoptotic cells by the catalysis of deoxyribonucleotide terminal transferase, and then is also bound to the luciferin antibody linked with horseradish peroxidase, and thus, the apoptotic cells can be accurately positioned through the bond of the luciferin antibody with the horseradish peroxidase substrate diaminobenzidine.

Specifically, at 3 days, 7 days, 14 days, and 28 days after drug injection, the tissues at the injection site were cut, pretreated, added with 2% hydrogen peroxide in PBS in the color cylinder, reacted at room temperature for 5 min, and washed with PBS; the tissue sections were added with 2 drops of TDT enzyme buffer solution after absorption of the excess liquid, and reacted at room temperature for 1-5 min; after carefully absorption of the excess liquid around the slice with filter paper, the tissue sections were immediately added with 54 μl TdT enzyme reaction solution, placed in a humid box and reacted at 37° C. for 1 hour; the tissue sections were placed in the staining jar, added with the stop reaction buffer preheated to 37° C., kept at 37° C. for 30 min, and the glass slides were gently lifted and put down every 10 minutes to slightly agitate the liquid; the tissue sections were washed 3 times with PBS, and were directly dropwise added with two drops of peroxidase-labeled anti-digoxigenin antibody and reacted for 30 minutes at room temperature in the humidified box after 5 min per wash; the tissue sections were washed with PBS for 5 min, and the wash were repeated for 4 times; the tissue sections were directly dropwise added with freshly prepared 0.05% DAB solution, and developed at room temperature for 3˜6 minutes; the tissue sections were washed 4 times with distilled water, in which each wash of the first 3 times was performed for 1 min, and the wash of the last time was performed for 5 min; and the tissue sections were counterstained with methyl green for 10 min at room temperature. The tissue sections were washed 3 times with distilled water, in which the glass slides were lifted and put down 10 times in the wash of the first 2 times, and were let stand for 30 s in the wash of the last time; the tissue sections were wash three times with 100% n-butanol via the same method with above wash; the tissue sections were dehydrated with xylene for 2 min, repeated 3 times, and after mounting and drying, observation was performed via an optical microscope and the experimental results were recorded.

### 2.3.4 Computer Image Analysis

The processed tissue sections were sorted according to the number, and observed through an optical microscope. The changes in the number of blood vessels in each tissue section were observed under the adjusted high-power field of view (DABX400), and the changes in the number of apoptosis were observed by the TUNEL method. Five fields of view were randomly selected under the high-power field of view, and the image was analyzed using Tmage Proplus 6.0 software.

### 2.3.5 Statistical Analysis

All data were processed with SPSS statistical software, and the data were expressed as x±s. Two-way analysis of variance was used to compare the data between groups, and P<0.05 indicated that the difference was statistically significant.

### 3. Results

### 3.1 General Changes of Cockscomb After Drug Injection

Through continuous dynamic naked eye observation, the treated cocks had good overall conditions, eating had no abnormalities, and no deaths occurred during the observation period.

The treated cockscombs of the blank control group showed no abnormal changes after naked eye observation. Positive control group: white skin hills can be seen at the injection site after the drug injection; at 3th day after the injection, the drug injection site was dark red with mild edema; at 7th day after the injection, the drug injection site was light yellow and the edema decreased; at 14th day after the injection, the center part of the drug injection site had a black scab, the surrounding part was yellow, and the edema gradually decreased; at 28th day after the injection, the black scab at the injection site fell off, became light yellow, with no edema and with scar; and the appearance of the cockscomb remained normal throughout the experiment. The overall condition of the groups A-I of the pharmaceutical composition was similar to that of the positive control group. At 14th days after the injection, part of the black scab fell off; at 28th days after the injection, the black scab fell off, with no obvious edema and scars; and compared with the groups D-I, the groups A-C had a faster onset time, and the effect of drugs on the cockscomb at each time point is greater.

The details of the general changes of cockscombs are shown in Table 2 to Table 4 below.

TABLE 2
Cockscomb appearance after drug injection

| | 0 d | 3 d | 7 d | 14 d | 28 d |
|---|---|---|---|---|---|
| blank control group | normal | normal | normal | normal | normal |
| Positive control group | normal | normal | normal | normal | normal |
| Group A | normal | normal | normal | normal | normal |
| Group B | normal | normal | normal | normal | normal |
| Group C | normal | normal | normal | normal | normal |
| Group D | normal | normal | normal | normal | normal |
| Group E | normal | normal | normal | normal | normal |
| Group F | normal | normal | normal | normal | normal |
| Group G | normal | normal | normal | normal | normal |
| Group H | normal | normal | normal | normal | normal |
| Group I | normal | normal | normal | normal | normal |

TABLE 3
The color and scab at the cockscomb injection site after drug injection

| | 0 d | 3 d | 7 d | 14 d | 28 d |
|---|---|---|---|---|---|
| blank | white | same color | same color | same color with the | same color with |
| control | skin | with the | with the | surrounded tissue | the surrounded |
| group | hill | surrounded | surrounded | | tissue |
| | | tissue | tissue | | |
| Positive | white | dark red | light yellow | black scab, yellow | black scab fell |
| control | skin | | | surrounding part | off, light yellow |
| group | hill | | | | |
| Group | white | dark red | light yellow | black scab, partly | black scab fell |
| A | skin | | | fell off, yellow | off, light yellow |
| | hill | | | surrounding part | |
| Group | white | dark red | light yellow | black scab, partly | black scab fell |
| B | skin | | | fell off, yellow | off, light yellow |
| | hill | | | surrounding part | |
| Group | white | dark red | light yellow | black scab, partly | black scab fell |
| C | skin | | | fell off, yellow | off, light yellow |
| | hill | | | surrounding part | |
| Group | white | slightly | slightly light | mild black scab | part of black scab |
| D | skin | darker than | yellow | | fell off, yellow |
| | hill | the | | | surrounding part |
| | | surrounding | | | |
| Group | white | slightly | slightly light | mild black scab | part of black scab |
| E | skin | darker than | yellow | | fell off, yellow |
| | hill | the | | | surrounding part |
| | | surrounding | | | |
| Group | white | slightly | slightly light | mild black scab | part of black scab |
| F | skin | darker than | yellow | | fell off, yellow |
| | hill | the | | | surrounding part |
| | | surrounding | | | |
| Group | white | slightly | slightly light | mild black scab | part of black scab |
| G | skin | darker than | yellow | | fell off, yellow |
| | hill | the | | | surrounding part |
| | | surrounding | | | |
| Group | white | slightly | slightly light | mild black scab | part of black scab |
| H | skin | darker than | yellow | | fell off, yellow |
| | hill | the | | | surrounding part |
| | | surrounding | | | |
| Group | white | slightly | slightly light | mild black scab | part of black scab |
| I | skin | darker than | yellow | | fell off, yellow |
| | hill | the | | | surrounding part |
| | | surrounding | | | |

TABLE 4
Cockscomb edema after drug injection

| | 0 d | 3 d | 7 d | 14 d | 28 d |
|---|---|---|---|---|---|
| blank | no | no | no | no | no |
| control | | | | | |
| group | | | | | |
| Positive | no | mild edema | edema | faintly edema | no edema, scar |
| control | | | decrease | | |
| group | | | | | |
| Group A | no | mild edema | mild edema | edema | no edema and |
| | | | | decrease | scar |
| Group B | no | mild edema | mild edema | edema | no edema and |
| | | | | decrease | scar |
| Group C | no | mild edema | mild edema | edema | no edema and |
| | | | | decrease | scar |
| Group D | no | no obvious | no obvious | no obvious | mild edema |
| | | edema | edema | edema | |
| Group E | no | no obvious | no obvious | no obvious | mild edema |
| | | edema | edema | edema | |
| Group F | no | no obvious | no obvious | no obvious | mild edema |
| | | edema | edema | edema | |
| Group G | no | no obvious | no obvious | no obvious | mild edema |
| | | edema | edema | edema | |
| Group H | no | no obvious | no obvious | no obvious | mild edema |
| | | edema | edema | edema | |
| Group I | no | no obvious | no obvious | no obvious | mild edema |
| | | edema | edema | edema | |

### 3.2 Histopathological Changes Under Light Microscope in Cockscomb After Drug Injection

Through the observation via a light microscope, it showed that in the blank control group, epithelial cells were neatly arranged, subcutaneous capillaries were abundant, morphology was regular, loose connective tissue and adipose tissue were present between the blood vessels, and some collagen fibers were included. After the drug injection, the overall trend of each group was that the capillaries gradually decreased, the collagen fibers gradually increased, and the changes of inflammatory cell infiltration and interstitial edema appeared in the early stage. In the positive control group, subcutaneous capillaries were destroyed and fused 3 days after injection, inflammatory cells in the peripheral tissues were infiltrated, interstitial edema was obvious, and a small amount of collagen fibers proliferated; 7 days later, capillaries were further decreased, collagen fibers were further proliferated, and inflammatory cells infiltrated and interstitial edema decreased; 14 days later, the number of blood vessels continued to decrease, and collagen fibers continued to proliferate; after 28 days, the capillaries were significantly reduced, and they were filled with a large number of collagen fibers. The overall situation of each of the pharmaceutical composition of groups A-I was similar to that of the positive control group; and compared with the groups D-I, the decrease of the number of capillaries and the proliferation of collagen fibers in the groups A-C is more obvious.

The details of the pathological changes of cockscombs are shown in Tables 5-7 below.

TABLE 5
Cockscomb epithelial cells after drug injection

| Group | 3 d | 7 d | 14 d | 28 d |
|---|---|---|---|---|
| blank control | neatly arranged | neatly arranged | neatly arranged | neatly arranged |
| group | | | | |
| Positive | neatly and tightly | neatly and | epithelial | epithelial |
| control group | arranged | tightly arranged | integrity, | integrity, |
| | | | basement | basement |
| | | | membrane | membrane |
| | | | thickening | thickening |
| Group A | neatly and tightly | neatly and | epithelial | epithelial |
| | arranged | tightly arranged | integrity, | integrity, |
| | | | basement | basement |
| | | | membrane | membrane |
| | | | thickening | thickening |
| Group B | neatly and tightly | neatly and | epithelial | epithelial |
| | arranged | tightly arranged | integrity, | integrity, |
| | | | basement | basement |
| | | | membrane | membrane |
| | | | thickening | thickening |
| Group C | neatly and tightly | neatly and | epithelial | epithelial |
| | arranged | tightly arranged | integrity, | integrity, |
| | | | basement | basement |
| | | | membrane | membrane |
| | | | thickening | thickening |
| Group D | neatly and tightly | neatly and | neatly and | neatly and tightly |
| | arranged | tightly arranged | tightly arranged | arranged |
| Group E | neatly and tightly | neatly and | neatly and | neatly and tightly |
| | arranged | tightly arranged | tightly arranged | arranged |
| Group F | neatly and tightly | neatly and | neatly and | neatly and tightly |
| | arranged | tightly arranged | tightly arranged | arranged |
| Group G | neatly and tightly | neatly and | neatly and | neatly and tightly |
| | arranged | tightly arranged | tightly arranged | arranged |
| Group H | neatly and tightly | neatly and | neatly and | neatly and tightly |
| | arranged | tightly arranged | tightly arranged | arranged |
| Group I | neatly and tightly | neatly and | neatly and | neatly and tightly |
| | arranged | tightly arranged | tightly arranged | arranged |

TABLE 6
The morphology and number of subcutaneous capillaries of cockscomb after drug injection

| Group | 3 d | 7 d | 14 d | 28 d |
|---|---|---|---|---|
| blank | abundant, | abundant, | abundant, regular | abundant, regular |
| control | regular | regular | morphology | morphology |
| group | morphology | morphology | | |
| Positive | some were | some were | the number was | the number were |
| control | destroyed and | further | significantly | further decreased, |
| group | fused | destroyed and | decreased, the | and most of the |
| | | fused, and some | lumens was | lumens disappeared |
| | | of lumens were | significantly | |
| | | narrowed | narrowed, and | |
| | | | some of the | |
| | | | lumens | |
| | | | disappeared | |
| Group A | some were | some were | the number was | the number were |
| | destroyed and | further | significantly | further decreased, |
| | fused | destroyed and | decreased, the | and some of lumens |
| | | fused, and some | lumens was | were significantly |
| | | of lumens were | significantly | narrowed |
| | | narrowed | narrowed, and | |
| | | | some of the | |
| | | | lumens | |
| | | | disappeared | |
| Group B | some were | some were | the number was | the number were |
| | destroyed and | further | significantly | further decreased, |
| | fused | destroyed and | decreased, the | and some of lumens |
| | | fused, and some | lumens was | were significantly |
| | | of lumens were | significantly | narrowed |
| | | narrowed | narrowed, and | |
| | | | some of the | |
| | | | lumens | |
| | | | disappeared | |
| Group C | some were | some were | the number was | the number were |
| | destroyed and | further | significantly | further decreased, |
| | fused | destroyed and | decreased, the | and some of lumens |
| | | fused, and some | lumens was | were significantly |
| | | of lumens were | significantly | narrowed |
| | | narrowed | narrowed, and | |
| | | | some of the | |
| | | | lumens | |
| | | | disappeared | |
| Group D | some were | some were | the number was | the number were |
| | destroyed and | further | decreased, and | decreased, lumens |
| | fused | destroyed and | some of the | were narrowed, and |
| | | fused, and | lumens were | some of lumens |
| | | partial lumens | narrowed | disappeared |
| | | were slightly | | |
| | | narrowed | | |
| Group E | some were | some were | the number was | the number were |
| | destroyed and | further | decreased, and | decreased, lumens |
| | fused | destroyed and | some of the | were narrowed, and |
| | | fused, and | lumens were | some of lumens |
| | | partial lumens | narrowed | disappeared |
| | | were slightly | | |
| | | narrowed | | |
| Group F | some were | some were | the number was | the number were |
| | destroyed and | further | decreased, and | decreased, lumens |
| | fused | destroyed and | some of the | were narrowed, and |
| | | fused, and | lumens were | some of lumens |
| | | partial lumens | narrowed | disappeared |
| | | were slightly | | |
| | | narrowed | | |
| Group G | some were | some were | the number was | the number were |
| | destroyed and | further | decreased, and | decreased, lumens |
| | fused | destroyed and | some of the | were narrowed, and |
| | | fused, and | lumens were | some of lumens |
| | | partial lumens | narrowed | disappeared |
| | | were slightly | | |
| | | narrowed | | |
| Group H | some were | some were | the number was | the number were |
| | destroyed and | further | decreased, and | decreased, lumens |
| | fused | destroyed and | some of the | were narrowed, and |
| | | fused, and | lumens were | some of lumens |
| | | partial lumens | narrowed | disappeared |
| | | were slightly | | |
| | | narrowed | | |
| Group I | some were | some were | the number was | the number were |
| | destroyed and | further | decreased, and | decreased, lumens |
| | fused | destroyed and | some of the | were narrowed, and |
| | | fused, and | lumens were | some of lumens |
| | | partial lumens | narrowed | disappeared |
| | | were slightly | | |
| | | narrowed | | |

TABLE 7
The internal and external conditions of subcutaneous
capillary lumens of cockscomb after drug injection

| Group | 3 d | 7 d | 14 d | 28 d |
|---|---|---|---|---|
| blank control | loose connective | loose connective | loose | loose connective |
| group | tissue and adipose | tissue and | connective | tissue and adipose |
| | tissue were | adipose tissue | tissue and | tissue were |
| | present between | were present | adipose tissue | present between |
| | the blood vessels, | between the | were present | the blood vessels, |
| | and some collagen | blood vessels, | between the | and some |
| | fibers were | and some | blood vessels, | collagen fibers |
| | included | collagen fibers | and some | were included |
| | | were included | collagen fibers | |
| | | | were included | |
| Positive | some of the | blood cells | the distance | the distance |
| control group | lumens filled with | gathered in | between blood | between blood |
| | clumps of blood | some lumens, | vessels was | vessels was |
| | cells, | inflammatory | enlarged, the | significantly |
| | inflammatory | cell infiltration | inflammation | enlarged, the |
| | cells in the | in peripheral | and edema are | inflammation and |
| | peripheral tissues | tissues | not obvious, | edema reaction |
| | were obviously | decreased, | and the | were not obvious, |
| | infiltrated, | interstitial | collagen fibers | and a large |
| | interstitial edema | edema was | are further | number of |
| | was obvious, and | relieved, and | proliferated | collagen fibers |
| | a small amount of | collagen fiber | | were filled |
| | collagen fibers | proliferation | | |
| | proliferated | was obvious | | |
| Group A | some of the | blood cells | the distance | the distance |
| | lumens filled with | gathered in | between blood | between blood |
| | clumps of blood | some lumens, | vessels was | vessels was |
| | cells, | inflammatory | enlarged, mild | significantly |
| | inflammatory | cell infiltration | inflammation | enlarged, the |
| | cells in the | in peripheral | and edema | inflammation and |
| | peripheral tissues | tissues | reaction | edema reaction |
| | were infiltrated, | decreased, | occurred, and | were not obvious, |
| | interstitial edema | interstitial | collagen fibers | and a large |
| | was presented, | edema was | were further | number of |
| | and a small | relieved, and | proliferated | collagen fibers |
| | amount of | collagen fiber | | were filled |
| | collagen fibers | proliferation | | |
| | proliferated | was obvious | | |
| Group B | some of the | blood cells | the distance | the distance |
| | lumens filled with | gathered in | between blood | between blood |
| | clumps of blood | some lumens, | vessels was | vessels was |
| | cells, | inflammatory | enlarged, mild | significantly |
| | inflammatory | cell infiltration | inflammation | enlarged, the |
| | cells in the | in peripheral | and edema | inflammation and |
| | peripheral tissues | tissues | reaction | edema reaction |
| | were infiltrated, | decreased, | occurred, and | were not obvious, |
| | interstitial edema | interstitial | collagen fibers | and a large |
| | was presented, | edema was | were further | number of |
| | and a small | relieved, and | proliferated | collagen fibers |
| | amount of | collagen fiber | | were filled |
| | collagen fibers | proliferation | | |
| | proliferated | was obvious | | |
| Group C | some of the | blood cells | the distance | the distance |
| | lumens filled with | gathered in | between blood | between blood |
| | clumps of blood | some lumens, | vessels was | vessels was |
| | cells, | inflammatory | enlarged, mild | significantly |
| | inflammatory | cell infiltration | inflammation | enlarged, the |
| | cells in the | in peripheral | and edema | inflammation and |
| | peripheral tissues | tissues | reaction | edema reaction |
| | were infiltrated, | decreased, | occurred, and | were not obvious, |
| | interstitial edema | interstitial | collagen fibers | and a large |
| | was presented, | edema was | were further | number of |
| | and a small | relieved, and | proliferated | collagen fibers |
| | amount of | collagen fiber | | were filled |
| | collagen fibers | proliferation | | |
| | proliferated | was obvious | | |
| Group D | inflammatory | peripheral tissue | peripheral | the distance |
| | cells in peripheral | inflammation | tissue | between blood |
| | tissue were | and edema were | inflammation | vessels was |
| | slightly infiltrated, | not obvious, and | and edema | enlarged, |
| | mild interstitial | collagen fibers | were not | inflammation and |
| | edema occurred, | were further | obvious, and | edema were not |
| | and a small | proliferated | collagen fibers | obvious, and |
| | amount of | | were obviously | collagen fibers |
| | collagen fiber | | proliferated | were obviously |
| | proliferated | | | proliferated |
| Group E | inflammatory | peripheral tissue | peripheral | the distance |
| | cells in peripheral | inflammation | tissue | between blood |
| | tissue were | and edema were | inflammation | vessels was |
| | slightly infiltrated, | not obvious, and | and edema | enlarged, |
| | mild interstitial | collagen fibers | were not | inflammation and |
| | edema occurred, | were further | obvious, and | edema were not |
| | and a small | proliferated | collagen fibers | obvious, and |
| | amount of | | were obviously | collagen fibers |
| | collagen fiber | | proliferated | were obviously |
| | proliferated | | | proliferated |
| Group F | inflammatory | peripheral tissue | peripheral | the distance |
| | cells in peripheral | inflammation | tissue | between blood |
| | tissue were | and edema were | inflammation | vessels was |
| | slightly infiltrated, | not obvious, and | and edema | enlarged, |
| | mild interstitial | collagen fibers | were not | inflammation and |
| | edema occurred, | were further | obvious, and | edema were not |
| | and a small | proliferated | collagen fibers | obvious, and |
| | amount of | | were obviously | collagen fibers |
| | collagen fiber | | proliferated | were obviously |
| | proliferated | | | proliferated |
| Group G | inflammatory | peripheral tissue | peripheral | the distance |
| | cells in peripheral | inflammation | tissue | between blood |
| | tissue were | and edema were | inflammation | vessels was |
| | slightly infiltrated, | not obvious, and | and edema | enlarged, |
| | mild interstitial | collagen fibers | were not | inflammation and |
| | edema occurred, | were further | obvious, and | edema were not |
| | and a small | proliferated | collagen fibers | obvious, and |
| | amount of | | were obviously | collagen fibers |
| | collagen fiber | | proliferated | were obviously |
| | proliferated | | | proliferated |
| Group H | inflammatory | peripheral tissue | peripheral | the distance |
| | cells in peripheral | inflammation | tissue | between blood |
| | tissue were | and edema were | inflammation | vessels was |
| | slightly infiltrated, | not obvious, and | and edema | enlarged, |
| | mild interstitial | collagen fibers | were not | inflammation and |
| | edema occurred, | were further | obvious, and | edema were not |
| | and a small | proliferated | collagen fibers | obvious, and |
| | amount of | | were obviously | collagen fibers |
| | collagen fiber | | proliferated | were obviously |
| | proliferated | | | proliferated |
| Group I | inflammatory | peripheral tissue | peripheral | the distance |
| | cells in peripheral | inflammation | tissue | between blood |
| | tissue were | and edema were | inflammation | vessels was |
| | slightly infiltrated, | not obvious, and | and edema | enlarged, |
| | mild interstitial | collagen fibers | were not | inflammation and |
| | edema occurred, | were further | obvious, and | edema were not |
| | and a small | proliferated | collagen fibers | obvious, and |
| | amount of | | were obviously | collagen fibers |
| | collagen fiber | | proliferated | were obviously |
| | proliferated | | | proliferated |

### 3.3 Results of Decrease of Cockscomb Capillaries at Different Time Points After Drug Injection

The capillary decrease of cockscomb was observed at the four time points of 3 d, 7 d, 14 d, 28 d after drug injection, as shown in table 8. Each group showed a trend of gradual increase in capillary decrease, and the capillary reduction in each group was more obvious in the first 7 days, After 7 days, the decrease of the capillary slowed down. Compared with the blank control group, the groups A-I significantly reduced the number of capillaries, reached a significant difference P<0.05 after 7 days, and reached a significant difference P<0.01 after 14 days. Especially for groups A-C, a significant difference P<0.05 was reached after 3 days, and a significant difference P<0.01 was reached after 7 days.

The decrease in the number of capillaries is basically consistent with the results of visual inspection and histopathology of the cockscomb. The details of the results obtained through 2.3.2 HE staining and 2.3.4 computer image analysis are shown in Table 8 below.

TABLE 8
Changes in the number of capillaries in each
group after treatment with cockscombs

| Group | 3 d | 7 d | 14 d | 28 d |
|---|---|---|---|---|
| blank | 57.65 ± 3.89 | 58.78 ± 3.70 | 56.76 ± 3.52 | 57.38 ± 4.01 |
| control | | | | |
| group | | | | |
| Positive | 47.05 ± 4.12 | 41.89 ± 4.25 | 36.59 ± 3.62 | 24.68 ± 4.33 |
| control | | | | |
| group | | | | |
| Group A | 46.56 ± 3.86 | 40.75 ± 4.13 | 35.81 ± 3.95 | 23.43 ± 4.21 |
| Group B | 47.31 ± 3.27 | 41.73 ± 4.09 | 35.97 ± 3.83 | 24.82 ± 3.86 |
| Group C | 46.83 ± 3.91 | 40.89 ± 3.95 | 36.01 ± 4.02 | 23.67 ± 4.19 |
| Group D | 51.91 ± 3.54 | 47.56 ± 3.93 | 41.29 ± 3.67 | 32.45 ± 3.79 |
| Group E | 49.32 ± 4.03 | 46.84 ± 4.45 | 40.31 ± 4.05 | 33.03 ± 4.19 |
| Group F | 50.69 ± 3.89 | 47.32 ± 3.89 | 41.43 ± 3.58 | 33.32 ± 3.68 |
| Group G | 50.35 ± 3.86 | 46.06 ± 4.23 | 41.09 ± 3.87 | 32.95 ± 3.91 |
| Group H | 50.52 ± 3.91 | 47.66 ± 4.06 | 41.35 ± 3.86 | 32.85 ± 4.06 |
| Group I | 51.32 ± 4.05 | 46.89 ± 3.81 | 40.59 ± 4.07 | 33.02 ± 3.82 |

### 3.4 Changes in Vascular Endothelial Cell Apoptosis After Drug Injection

Through continuous dynamic observation via an optical microscope, it showed that there was almost no apoptosis in the blank control group. After injection of the composition 1-9 of the disclosure, a large number of apoptotic vascular endothelial cells can be seen, and the number of apoptotic vascular endothelial cells increased over time. Compared with the groups D-I, the number of apoptotic cells in the groups A-C was significantly higher, even comparable to the positive control group. Compared with the blank control group, the groups A-I reached a significant difference P<0.05 after 7 days, and reached a significant difference P<0.01 after 14 days. Especially for the groups A-C, a significant difference P<0.05 was reached after 3 days, and a significant difference P<0.01 was reached after 7 days.

The details of the results obtained through 2.3.3 cell apoptosis detection and 2.3.4 computer image analysis are shown in Table 9 below.

TABLE 9
Changes in the number of apoptotic cells after
treatment with cockscombs in each group

| Group | 3 d | 7 d | 14 d | 28 d |
|---|---|---|---|---|
| blank | 8.05 ± 2.62 | 8.35 ± 3.02 | 7.95 ± 2.78 | 8.18 ± 3.15 |
| control | | | | |
| group | | | | |
| Positive | 21.02 ± 3.56 | 36.78 ± 3.87 | 54.68 ± 3.65 | 68.62 ± 3.27 |
| control | | | | |
| group | | | | |
| Group A | 20.95 ± 3.56 | 36.85 ± 3.64 | 55.01 ± 3.78 | 67.98 ± 3.16 |
| Group B | 21.32 ± 3.49 | 36.90 ± 3.75 | 54.86 ± 3.82 | 69.02 ± 3.35 |
| Group C | 21.15 ± 3.77 | 36.62 ± 3.32 | 54.79 ± 3.97 | 68.71 ± 3.45 |
| Group D | 13.95 ± 2.56 | 23.07 ± 3.85 | 35.69 ± 3.74 | 55.38 ± 4.07 |
| Group E | 14.08 ± 2.84 | 22.86 ± 3.49 | 36.07 ± 3.58 | 56.06 ± 3.36 |
| Group F | 14.26 ± 2.57 | 22.48 ± 3.76 | 35.71 ± 3.78 | 55.45 ± 3.65 |
| Group G | 14.32 ± 2.31 | 22.53 ± 3.94 | 35.78 ± 3.27 | 55.83 ± 3.79 |
| Group H | 13.96 ± 2.68 | 22.67 ± 3.81 | 35.84 ± 3.81 | 55.58 ± 3.83 |
| Group I | 14.65 ± 2.84 | 22.77 ± 3.83 | 35.91 ± 3.86 | 55.64 ± 3.73 |

### 4. Conclusion

Each of the pharmaceutical compositions 1-9 of the disclosure can have significant effects on cockscombs, such as reducing the number of capillaries, proliferating collagen fibers, and apoptotic vascular endothelial cells. Especially, for the cockscomb hemangioma model, the compositions 1-3 have better effects in the disappearance time of the vascular wall structure, damage of the vascular endothelium and induction of the apoptosis of capillary endothelial cells, and the decrease of the number of capillaries, and will not change the shape of the cockscomb. Therefore, the combined use of citric acid, paeoniflorin, and gallic acid produces a synergistic effect, especially when the weight ratio of citric acid, paeoniflorin, and gallic acid is about 100:(2.2˜5.7):(1˜2), and when the weight ratio of paeoniflorin and gallic acid is about (2.31˜2.89):1.

### Example 2

### Clinical Example

Clinical Case 1: Treatment of Rectal Hemangioma

Patient: Mr. Li, male, 22 years old.

The main symptoms: hematochezia, anemia and blood in the stool. The blood in the stool was persistent, and the bleeding was dark red or bright red. Fiber colonoscopy was performed before treatment, and it was characterized by ring-shaped, irregular purple bumps in the rectum, smooth mucosal surface, mucosal erosion accompanied by active bleeding, and thus, he was diagnosed with a rectal hemangioma.

Treatment method: The parenteral solution (concentration 2.635%) of the pharmaceutical composition 1 of the disclosure and 0.5% lidocaine were prepared as a 1:1 mixture according to the volume ratio. The patient took the folding knife position, the protective film pulled both buttocks to expose the anus, the anus automatic retractor was used to expose the lesion area, the normal rectal mucosa at 0.5 cm from the edge of the hemangioma was punctured to the muscle layer with a No. 7 puncture needle in the direction of the center of the hemangioma, and when there was no blood in the withdrawal, the above-prepared mixed solution was injected at several points along the edge of the hemangioma, and a total of about 5 mL of the mixed solution was injected.

Results: Three months after the injection, the symptoms of rectal hemangioma disappeared completely, the lesions were smooth and the effect was good. During the one-year follow-up, there was no adverse reaction symptom and no trace of the lesion.

Clinical Case 2: Hemophilia with Hemorrhoid

Patient: Mr. Wan, male, 17 years old.

The main symptoms: the perianal mass prolapsed after defecation, and can be absorbed by itself, accompanied by bleeding for two days, each bleeding about 3 mL, the local clinic prescribed oral medications, and the symptoms were relieved, but the symptoms were not controlled.

After inspection:

(1) T37.0° C. P78 times/min R19 times/min BP110/70 mmHg, no obvious abnormality was found in the physical examination;

(2) Specialist examination: anal lithotomy position: anal margin was not flat, anal canal mucosa bulged across the tooth line at 3, 7, and 11 o'clock under an anoscope; digital rectal examination: no other masses were touched in the lower rectum, and no blood stains were present in finger cuffs when withdrawing;

(3) The activity of factor VIII was 121.2%, the activity of factor IX was 9.2%, and the activity of factor X was 107.2%.

He was diagnosed with hemophilia with hemorrhoid. The risk of bleeding was high and surgery cannot be performed. Therefore, the injection of the pharmaceutical composition 1 of the disclosure was used.

Treatment method: conventional disinfection, or anal canal anesthesia was performed. The parenteral solution of the pharmaceutical composition 1 of the disclosure (concentration 2.635%) and 0.5% lidocaine were prepared as a 1:1 mixture according to the volume ratio, and the hemorrhoid was exposed under anoscope, and a needle was obliquely punctured at the bulged part of the hemorrhoid surface, the appropriate injection volume for each injection was that the hemorrhoid were uniform, plump, and filled, and the surface mucosa was pink, and the total injection was about 10 mL.

Result: there was no bleeding after the operation, and he was discharged from the hospital three days later. After three months of re-examination, no adverse reaction symptoms were seen, the lesion was flat, hemorrhoid fell off, and there was no abnormality in the lesion. Followed up for one year, there was no recurrence.

The above are only specific embodiments of the disclosure. It should be pointed out that for those of ordinary skill in the art, improvements can be made to the composition and method of the disclosure without departing from the concept of the disclosure. All improvements shall belong to the protection scope of the disclosure.

What is claimed is:

1. A pharmaceutical composition containing citric acid, paeoniflorin and gallic acid in a weight ratio of about 100:(1˜8):(0.1˜5), wherein a weight ratio of paeoniflorin to gallic acid is about (1˜2.63):1 or (2.89˜5):1.

2-5. (canceled)

6. A preparation comprising the pharmaceutical composition of claim 1 and a pharmaceutically acceptable excipient.

7. The preparation of claim 6, which is an injection; and comprises the pharmaceutical composition and water for injection.

8-9. (canceled)

10. A use of the pharmaceutical composition of claim 1 in the preparation of a drug for treating rectal hemangioma or hemorrhoid.

11. The use of claim 10, wherein the drug is used for a patient who is not suitable for surgery.

12. The use of claim 11, wherein the patient who is not suitable for surgery includes a patient with bleeding tendency, hypertension, diabetes, portal hypertension, renal dysfunction, myasthenia gravis, or immunodeficiency, or a patient who is receiving chemoradiotherapy.

13. The use of claim 12, wherein the patient with bleeding tendency includes a patient with coagulation dysfunction, or a patient receiving antithrombotic therapy; and wherein the patient with coagulation dysfunction includes a patient with aplastic anemia, leukemia or hemophilia, and
the patient receiving antithrombotic therapy includes a patient receiving cardiac stent surgery or cardiac pacemaker surgery, or a patient with myocardial infarction or cerebral infarction.

14. The use of claim 12, wherein the patient with immunodeficiency includes a patient with AIDS or systemic lupus erythematosus.

15-23. (canceled)

24. A method for treating rectal hemangioma or hemorrhoid in a patient, including administering the preparation of claim 6.

25. The method of claim 24, wherein the patient is a patient who is not suitable for surgery.

26. The method of claim 25, wherein the patient who is not suitable for surgery includes a patient with bleeding tendency, hypertension, diabetes, portal hypertension, renal dysfunction, myasthenia gravis, or immunodeficiency, or a patient who is receiving chemoradiotherapy.

27. The method of claim 26, wherein the patient with bleeding tendency includes a patient with coagulation dysfunction, or a patient receiving antithrombotic therapy, wherein the patient with coagulation dysfunction includes a patient with aplastic anemia, leukemia or hemophilia, and
the patient receiving antithrombotic therapy includes a patient receiving cardiac stent surgery or cardiac pacemaker surgery, or a patient with myocardial infarction or cerebral infarction.

28. The method of claim 26, wherein the patient with immunodeficiency includes a patient with AIDS and systemic lupus erythematosus.

29-30. (canceled)

31. The method of claim 24, wherein an administered preparation is an injection in water for injection, and the injection comprises the pharmaceutical composition and water for injection, and wherein a drug concentration is 22-30 mg/mL.

32. The method of claim 31, wherein in the injection, a volume ratio of the pharmaceutical composition and water for injection is 1:(0.8˜1.2).

33. The method of claim 31, wherein the injection is injected to the patient 1-3 times.

34. The method of claim 31, including a following steps:
(1) Placing the patient at a side lying position, a folding knife position and a bladder lithotomy position and other treatment positions, performing routine disinfection, and then anal local anesthesia or anal canal anesthesia with 0.5-1% of lidocaine;
(2) When using the injection, diluting the injection with 0.5-1% of lidocaine at a volume ratio of the injection to lidocaine being 1:(0.8˜1.2);
(3) For stage I and II internal hemorrhoid and varicose mixed hemorrhoid, exposing each hemorrhoid under an anoscope, and inserting a needle into the hemorrhoid obliquely;
(4) Retracting the anoscope to expose the hemorrhoid, in which an injection method for stage III internal hemorrhoid is the same as that for stage I and II internal hemorrhoid;
(5) Administering 10-20 ml to each patient at a time, with an average of 15 ml and a maximum dosage of no more than 40 ml; and
(6) Generally giving only one injection to each patient.

35. (canceled)
